(19) United States
(12) Patent Application Publication
(10) Pub. No.: US 2004/0127551 A1
(43) Pub. Date: Jul. 1, 2004

(54) Taxane-based compositions and methods of use

(76) Inventors: Kai Zhang, Miami, FL (US); Gregory A. Smith, North Miami, FL (US)

Correspondence Address: IVAX CORPORATION, 4400 Biscayne Boulevard, Miami, FL, 33137, US

(21) Appl. No.: 10/330,804

(22) Filed: Dec. 27, 2002

(51) Int. Cl. A61K 31/337; A61K 31/355

(52) U.S. Cl. 514/449; 514/458

(57) ABSTRACT

Disclosed are taxane-based compositions and methods of using the same to achieve target blood levels of a taxane in a mammal, e.g., to treat taxane-responsive malignant and non-malignant diseases. Compositions of the invention exhibit long-term stability and overall palatability. Also disclosed are methods for using the compositions as analytical tools for pharmacokinetic studies.

### FIELD OF THE INVENTION

The invention relates to novel compositions useful to administer aqueous insoluble medicaments, including medicaments known to be poorly absorbed when administered orally. The invention further relates to compositions and methods of using the same, to achieve target blood levels of a taxane in a mammal. Moreover, the invention relates to methods of treatment employing such compositions.

### BACKGROUND OF THE INVENTION

The scarcity of effective approaches to address poor solubility characteristic of many pharmacologically useful compounds (e.g., lipophilic, hydrophobic and amphiphobic compounds) is a critical shortcoming hindering drug development. Kagkadis et al., PDA J. Pharm. Sci. 50(5):317-323 (1996) and Sweetana et al., PDA Pharm. Sci. 50(5):330-342 (1996) teach that poorly soluble compounds include for example cortisone, etoposide, cyclosporin and proleukin. Traditionally, because of poor or inconsistent systemic absorption from the gastrointestinal tract, poorly soluble drugs have been administered intravenously (involving considerable physical and psychological discomfort and potential local trauma, as well as additional economic costs).

Poorly soluble chemotherapeutic and/or anticancer agents include taxanes, such as paclitaxel, which are not normally bioavailable when administered orally. Wani et al., J. Am. Chem. Soc., 93:2325 (1971) teaches that paclitaxel, a member of the taxane family of terpenes, is a natural diterpene product isolated from the Pacific yew tree (Taxus brevifolia). Although the exact mechanism responsible for paclitaxel's chemotherapeutic properties has not been elucidated, several studies, such as those of Schiff et al., Proc. Natl. Acad. Sci. USA, 77:1561-1565 (1980); Schiff et al., Nature, 277:665-667 (1979); and Kumar, J. Biol. Chem., 256:10435-10441 (1981), postulate that paclitaxel's ability to inhibit tumorigenic growth stems from its capacity to bind the N-terminal 31 amino acids of the beta-tubulin subunit in the microtubule (see Rao et al., J Biol Chem 269:3132-3134 (1994). Wood et al., New Eng. J. M. 332(15):1004-1014 (1995) attributes paclitaxel anticancer properties to the inhibition of disassembly of microtubules rendering them extraordinarily stable and dysfunctional, thereby causing cell death by disrupting normal dynamics required during cell division and vital interphase processes.

The scientific literature is replete of papers reporting the efficacy of paclitaxel in the treatment of a variety of unrelated conditions. See for example, Einzig et al., Proc. Am. Soc. Clin. Oncol., 20:46 (1996) for lung cancer and head and neck carcinomas; Forastire et al., Sem. Oncol., 20:56 (1990) for neoplasms in the skin; Chang et al., Cancer 77(1):14-18 (1996) for gastric cancer); Woo et al., Nature, 368:750 (1994) for polycystic kidney disease; and Pouvelle et al., J. Clin. Invest. 44:413-417 (1994) for malaria.

Paclitaxel and docetaxel have been approved for clinical use in the treatment of several, unrelated conditions. Markman et al., Yale J. of Bio. & Med., 64:583 (1991), and McGuire, et al., Ann. Intern. Med. 111:273 (1989) disclose the use of paclitaxel for refractory ovarian cancer in the United States; Mavrodius et al., ASCO 18:254a (1999) describes the use of docetaxel for gastric cancer; Holmes et al., J. Nat. Cancer Inst., 83:1797 (1991) discloses the use of paclitaxel for chemotherapy for several types of neoplasms including breast cancer (see also Taxol (paclitaxel) Mead Johnson Oncology Products package insert); Fencel et al., ASCO 18:283a (1999) teaches the use of paclitaxel and docetaxel for esophageal cancer; Vanhoefer et al., ASCO 18:303a (1999) describes phase II studies using docetaxel in metastatic gastric cancer; Kourossis et al., ASCO 17:266(a) (1998) teaches the use of docetaxel as salvage chemotherapy for advanced gastric cancer; Xiao et al., ASCO 17:306(a) (1998) assessing new paclitaxel treatment regimens in patients with esophageal carcinoma who had been previously treated with paclitaxel; Schultz et al., reporting phase II trials of docetaxel in patients with hormone refractory prostate cancer; Ajani et al., J. Nat. Cancer Inst., 86:1086-1091 (1994), and Kelsen et al. Seminars in Oncology 21:44-48 (1994) describe paclitaxel regimens for squamous cell carcinoma and adenocarcinoma as well as epidermoid cancer of the esophagus.

Thus far, efforts have been directed to the development of (i) suitable injection and infusion taxane formulations and (ii) to more water-soluble taxane analogs, derivatives and prodrugs. Thus, most paclitaxel formulations for IV infusion have been developed utilizing polyethoxylated castor oil, commercially available as CREMOPHOR EL™, as the drug carrier. Polyethoxylated castor oil however, is itself toxic, produces vasodilation, labored breathing, lethargy, hypotension and death in dogs it is also suspected to cause allergic-type reactions when administered intravenously.

Alternative approaches have led to more water-soluble analogs, derivatives and prodrugs of taxanes. Hence, for example, “Modified Taxols IV; Synthesis and biological activity of taxols Modified in the side chain”, Magri, N. F.; Kingston, DGI; J. Nat. Prod 1988, 51, 298, teaches derivatized paclitaxel analogs in which the 2′ and/or 7-position is derivatized with groups that would enhance water solubility. These efforts have yielded prodrug compounds that are more water-soluble than the parent compound and that display the cytotoxic properties upon activation. One important group of such prodrugs includes the 2′-onium salts of paclitaxel and docetaxel (see e.g. Nicolaou, et al., Angew. Chin. Int. Engl. 33:1583-1587 (1994)), particularly the 2′-methylpyridinium mesylate (2′-MPM) salts disclosed in PCT publication no. WO 98/58927. Suffness (ed.) in Taxol® Science and Applications, CRC Press (1995) states that to date none has progressed to clinical evaluation because of marginal improvements in solubility, stability problems and low regeneration rates.

Preclinical studies have suggested that paclitaxel alone is not absorbed after oral doses. Walle et al., Drug Metabo. Disp. 26(4):343-346 (1998), reported that taxol is not absorbed after oral administration, and attributed low oral bioavailability to the action an outwardly directed efflux pump. Similarly, Eiseman, et al., Second NCI Workshop on Taxol and Taxus (September 1992), and Suffness (ed.) in Taxol® Science and Applications, CRC Press (1995) teach that paclitaxel is very poorly absorbed when administered orally (less than 1%). More specifically, Eiseman et al. indicates that paclitaxel has a bioavailability of 0% upon oral administration, and Suffness et al. reports that oral dosing with paclitaxel did not seem possible. For these reasons, paclitaxel has not been administered orally to human patients. Similarly, docetaxel (N-debenzoyl-N-tert-butoxycarbonyl-10-deacetyl paclitaxel), sold under the trademark TAXOTERE® (Rhone-Poulenc-Rorer S.A.) and administered in parenteral form for the treatment of breast cancer.

The poor bioavailability of paclitaxel after oral administration may be ascribed to a membrane-bound P-glycoprotein which functions as an energy-dependent transport, or efflux pump, to decrease intracellular accumulation of drug by extruding xenobiotics from the cell (see e.g., Taxol® Science and Applications, supra). It is hypothesized that, by preventing movement through mucosal cells of the small intestine, the P-glycoprotein prevents systemic absorption. A number of known agents have been shown to inhibit P-glycoprotein (e.g., cyclosporin A, verapamil, tamoxifen, quinidine and phenothiazines). Logically, efforts, including clinical trials, have been directed to study the effects of cyclosporine on anti-cancer agents known to be subject to multidrug resistance (MDR), such as paclitaxel (Fisher, et al., Proc. Am. Soc. Clin. Oncol. 13:143 1994); doxorubicin (Bartlett, et al., J. Clin. Onc. 12:835-842 (1994); and etoposide (Lum, et al., J. Clin. Onc. 10:1635-1642 (1992)). The intravenous administration of cyclosporine in conjunction with anti-cancer drugs has been shown to result in higher blood levels (presumably through reduced body clearance) and exhibited the expected toxicity at substantially lower dosage levels. For a general discussion of the pharmacologic implications for the clinical use of P-glycoprotein inhibitors, see Lum, et al., Drug Resist. Clin. Onc. Hemat. 9:319-336 (1995); Schinkel, et al., Eur. J. Cancer 31A: 1295-1298 (1995).

PCT publication WO 95/20980 (published Aug. 10, 1995) (hereinafter “Benet”) purports to teach a method for increasing the bioavailability of orally administered hydrophobic pharmaceutical compounds. This method comprises the concurrent oral administration of a bioenhancer including an inhibitor of a cytochrome P450 3A enzyme or an inhibitor of P-glycoprotein-mediated membrane transport. Benet does not identify which bioavailability enhancing agent(s) improve the availability of specific target pharmaceutical compounds, nor does it teach specific dosage amounts, schedules or regimens for administration of the enhancing or target agents. The only combination disclosed is ketoconazole as the enhancer, and cyclosporin A as the target drug.

Benet merely provides that bioenhancers are hydrophobic compounds generally comprising two co-planar aromatic rings, a positively charged nitrogen group or a carbonyl group—a class that includes an unascertainable number of compounds, including several inoperable embodiments. Moreover, the classes of active agents disclosed by Benet include the great majority of pharmaceutical agents listed in the Physicians' Desk Reference and thus, are of no value to medical practitioners seeking safe, practical and effective methods of orally administering specific agents. Finally, Benet provides no teaching that could be followed by one of skill to identify suitable bioenhancer/active drug combinations or to design therapeutically effective oral modalities.

PCT publication no. WO 98/30205 (published Jul. 16, 1998) (hereinafter “Quay”) allegedly discloses a method for increasing the bioavailability of poorly soluble drugs. The application discloses an emulsion of alpha-tocopherol including a surfactant. Also included is PEGylated Vitamin E. PEGylated alpha-tocopherol includes polyethylene glycol subunits attached by a succinic acid diester at the ring hydroxyl of Vitamin E. Alpha-tocopherol allegedly serves as a surfactant, stabilizer and a secondary solvent in emulsions of alpha-tocopherol. Notably, this reference is expressly limited to formulations that are (a) emulsions and (b) essentially ethanol-free.

Commonly-owned PCT publication no. WO 97/15269 discloses novel methods and compositions to make bioavailable target agents including taxanes otherwise displaying poor oral bioavailability by oral co-administration of a bioavailability-enhancing agent such as cyclosporin.

There remains a need to develop additional compositions and effective methods suitable for the oral administration of taxanes. Such compositions should be capable of achieving target therapeutic blood levels of taxane. For obvious practical reasons, such compositions should be (i) bioavailable, (ii) suitable to maintain the taxane in solution, (iii) chemically stable over extended periods of time and (iv) possess overall palatability while demonstrating long term stability.

### SUMMARY OF THE INVENTION

The present inventors have devised taxane-based compositions and methods of using the same useful to achieve target blood levels of the taxane, in a mammal. The compositions exhibit long-term stability and overall palatability. As exemplified herein, such approaches provide the means to achieve taxane blood levels comparable to the levels achieved by less convenient methodologies currently available such as, for example, therapeutically effective infusion modalities. The invention thus provides compositions and methods useful to improve the absorption of a taxane from the gastrointestinal tract into the bloodstream and to provide target blood levels, including therapeutically effective blood levels, of such taxane in a mammal. In some embodiments, the taxane blood levels achieved exceed those achieved by compositions disclosed in WO97/15269. Moreover, the methods and compositions according to the invention are useful as analytical tools for biochemical studies as well as therapeutic tools.

In a first aspect, the invention provides pharmaceutical compositions demonstrating long-term stability and overall palatability. Such compositions comprise a poorly soluble medicament, a carrier, a co-solubilizer, and a stabilizer. In a more preferred embodiment of the invention, the medicament is a taxane. Preferred embodiments of the invention may provide more than one type of taxane, carrier, co-solubilizer, or stabilizer. Some compositions of the invention further include additional components, such as for example surfactants, pharmaceutical excipients, diluents, sweeteners, flavoring agents or coloring agents, as described in more detail herein. In particularly preferred embodiments of the invention, the taxane is paclitaxel or docetaxel. Upon oral administration in conjunction with an oral bioavailability-enhancing agent, some of the preferred compositions of the invention provide taxane blood levels comparable to blood levels achieved by intravenous injection.

In a second aspect, the invention provides methods suitable to achieve therapeutically effective taxane blood levels in a mammal by the oral administration of the pharmaceutical compositions described. In particularly preferred embodiments, the methods of the invention, including the administration of a bioavailability enhancing agent, result in taxane blood levels which are comparable to those achieved by long term infusion such as 96-hours infusion shown to be therapeutically effective (e.g., for the treatment of advanced metastatic breast cancer as described in Wilson, et al., J. Clin. Oncol. 12:1621-1629 (1994), and Seidman, et al., J. Clin. Oncol. 16:3353-3361 (1998). Pharmaceutical compositions and bioavailability enhancing agents useful according to this aspect are as described for the first aspect of the invention.

In a third aspect, the invention provides a method to investigate the properties of diterpenoids. More specifically, the invention provides tools to investigate biochemical properties of taxane moieties in novel formulations capable of mediating large increases in solubility. Such studies will lead to a more comprehensive pharmacokinetic and pharmaceutical description of taxanes essential to identify novel applications and possibly to further optimize already existing therapeutic outcomes.

A further aspect of the invention pertains to methods of treatment of a mammal suffering from a taxane-responsive disease by the oral administration of pharmaceutical compositions as described herein. In some embodiments, the pharmaceutical compositions of the invention are orally administered in conjunction with an oral bioavailability-enhancing agent to provide blood levels of the taxane which are comparable to the levels achieved by intravenous injection of the taxane. Pharmaceutical compositions and bioavailability enhancing agents useful according to this aspect are as described for the first aspect according to the invention.

### BRIEF DESCRIPTION OF THE DRAWINGS

FIG. 1 is a graphic representation showing the ability of compositions of the invention (PG/TPGS/ETOH and ascorbyl palmitate (40:40:20) with (●) 12 mg/ml, (∇) 15 mg/ml, (▪) 20 mg/ml, (∘) 25 mg/ml, and (▾) 50 mg/ml paclitaxel), to remain in solution for a period of time≧2 hours in a reciprocal water-shaking bath.

FIG. 2 is a graphic representation showing the average plasma concentrations of paclitaxel from (●) 9 patients orally administered a Cremophor EL based formulation, and from (∘) 2 orally administered a composition of the present invention (PG/TPGS/ETOH and ascorbyl palmitate (40:40:20).

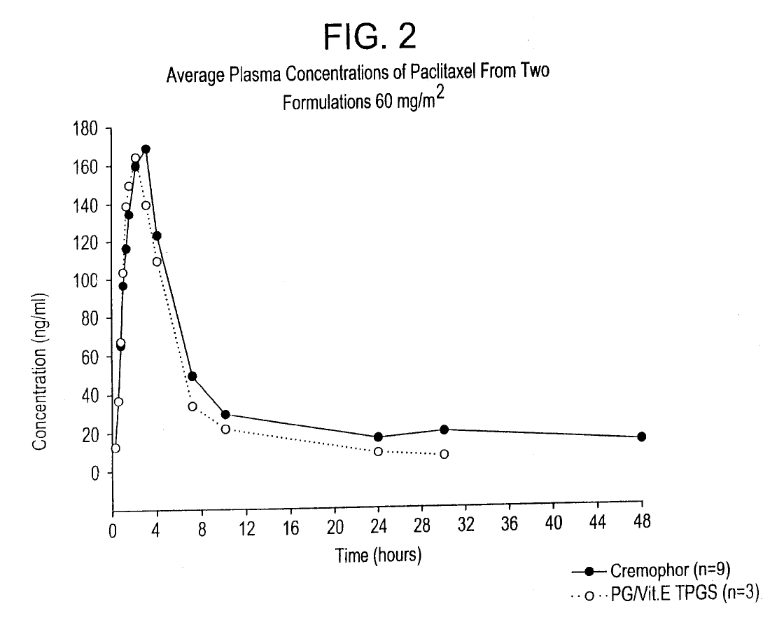

### DETAILED DESCRIPTION OF THE PREFERRED EMBODIMENTS

The present inventors have devised novel compositions and methods of using the same to orally administer aqueous insoluble medicaments, including medicaments known to be poorly absorbed when administered orally. The invention further relates to compositions and methods of using the same useful to achieve target blood levels, including therapeutic blood levels, of a taxane in a mammal. Moreover, the invention relates to treatment regimens employing such compositions. The U.S. patents and other publications identified herein are within the knowledge of those skilled in this field and are hereby incorporated by reference in their entirety.

Technical and scientific terms used herein have the meaning commonly understood by one of skill in the art to which the present invention pertains, unless otherwise defined. To ensure a clear and complete understanding of the specification and claims, including the scope to be given such terms, the following definitions are provided. It is understood that terms as defined may appear in the noun, verb, singular as well as the plural counterpart forms.

Reference is made herein to various methodologies and materials known to those of skill in the art. Standard reference works setting forth the general principles of pharmacology include Goodman and Gilman's The Pharmacological Basis of Therapeutics, 9^{th} Ed., McGraw Hill Companies Inc., New York (1996). Standard reference works setting forth the general principles of modem pharmaceutics (Remington's Pharmaceutical Sciences, 18^{th} Ed., Gennaro, Mack Publishing Co., Easton, Pa. (1990) and Remington: The Science and Practice of Pharmacy, Lippincott, Williams & Wilkins (1995)).

Any suitable materials and/or methods known to those of skill can be utilized in carrying out the present invention. However, preferred materials and methods are described. Materials, reagents and the like to which reference is made in the following description and examples are obtainable from commercial sources, unless otherwise noted.

The present invention is intended for use with any mammal that may experience the benefits of the methods of the invention. Foremost among such mammals are humans, although the invention is not intended to be so limited, and is applicable to veterinary uses.

In a first aspect, the invention provides pharmaceutical compositions demonstrating long-term stability and overall palatability. Such compositions comprise a taxane, a carrier, a co-solubilizer, and a stabilizer. For purposes of the invention, the term “carrier” is used to denote a moiety that maintains (and in preferred embodiments improves) the aqueous solubility of the taxane in the pharmaceutical composition of the invention. Carriers according to the instant invention include without limitation moieties that may also function as co-solubilizers. The carriers of the invention are characterized by a core structure that may be either a straight chain polyether or a branched glycol (e.g., glycol) coupled with at least one fatty acid ester. Preferred carriers for use in the invention are non-ionic surfactants or emulsifiers having HLB values of at least about 10. It has been found that such non-ionic surfactants or emulsifiers are not only compatible carriers for the lipophilic taxanes (which are poorly soluble in water) but also promote absorption of the active ingredient from the gastrointestinal tract into the bloodstream. Only those members of these surfactant families that have HLB values of about 10 or greater may be used as carriers in the subject compositions.

Representative non-limiting examples of carriers according to the invention include Vitamin E TPGS (d-alpha-tocopheryl polyethylene glycol 1000 succinate, available from Eastman Chemical Co., Kingsport, Tenn.); saturated polyglycolyzed glycerides such as the GELUCIRE™ and LABRASOL™ products (Gattefossé Corp., Westwood, N.J.) which include glycerides of C₈-C₁₈ fatty acids; CREMOPHOR™ EL or other modified castor oils including polyoxyethylated or hydrogenated castor oils such as EL-P or RH40 modified castor oils (available from BASF, Mt. Olive, N.J.); MYRJ™ polyoxyethylated stearate esters (sold by ICI Americas, Charlotte, N.C.); TWEEN™ (ICI Americas) and CRILLET™ (available from Croda Inc., Parsippany, N.J.) polyoxyethylated sorbitan esters; BRIJ™ polyoxyethylated fatty ethers (ICI Americas); CROVOL™ modified (polyethylene glycol) almond and corn oil glycerides, including polyethylene glycol almond or corn oil glycerides (Croda Inc., Edison, N.J.); EMSORB™ sorbitan diisostearate esters (Henkel Corp., Ambler, Pa.); SOLUTOL™ polyoxyethylated hydroxystearates (BASF); and cyclodextrin.

Preferred pharmaceutical compositions of the invention comprise at least 30% by weight of carrier. In particularly preferred embodiments, the carrier is present in an amount of from about 30 to about 90% of the composition by weight. In a particularly preferred embodiment, the pharmaceutical composition of the invention comprises about 40% by weight of Vitamin E TPGS.

The term “co-solubilizer” is used to designate a viscosity-reducing moiety which increases the fluidity of the compositions of the invention at body temperature, as generally required for oral bioavailability, and/or reduce the melting point of the compositions below body temperature. Preferred co-solubilizers according to the invention decrease the viscosity and increase the fluidity of the vehicle at body temperature, and also may increase the amount of the active agent that can be dissolved or dispersed in the vehicle in comparison with the use of a carrier alone. Co-solubilizers according to the invention include moieties capable of functioning as carriers as well. Co-solubilizers according to the instant invention include without limitation moieties that may also provide increased taxane solubility.

Representative non-limiting examples of viscosity-reducing co-solubilizers include PHARMASOLVE™ (N-methyl-2-pyrrolidone, International Specialty Products, Wayne, N.J.); MIGLYOL™ glycerol or propylene glycol esters of caprylic and capric acids (Hüls AG, Marl, Germany); polyoxyethylated hydroxystearates, including stearyl or oleyl ethers (e.g., SOLUTOL™ HS 15) (BASF, Mt. Olive, N.J.); TWEEN™ polyoxyethylated sorbitan esters (ICI Wilmington, Del.); SOFTIGEN™ polyethylene glycol esters of caprylic and capric acids (Hüls AG); modified castor oils including polyoxyethylated or hydrogenated castor oils (such as CREMOPHOR™ EL, EP-P or RH 40) (BASF, Mt. Olive, N.J.); vegetable oils such as olive oil, polyoxyethylated fatty ethers or modified castor oils; certain saturated polyglycolyzed glycerides, including glycerides of C₈-C₁₈ fatty acids (such as a LABRASOL™); citrate esters such as tributyl citrate, triethyl citrate and acetyl triethyl citrate, propylene glycol, alone or in combination with PHARMASOLVE™, ethanol (preferably dehydrated ethanol), water, and lower molecular weight polyethylene glycols such as PEG 200, 300 and 400. In a particularly preferred embodiment, the co-solubilizer is ethanol. In a more particularly preferred embodiment, the co-solubilizer comprises propylene glycol and ethanol. Up to 90% of the composition by weight may be co-solubilizer. In some embodiments of the invention, from about 10 to about 70% by weight is co-solubilizer. In preferred embodiments of the invention, the co-solubilizer is present in an amount of from about 20 to about 60% by weight. Accordingly, preferred pharmaceutical compositions may comprise from about 10% to about 70% by weight of propylene glycol, more preferably from about 20 to about 60% by weight of propylene glycol. In a particularly preferred embodiment the pharmaceutical composition of the invention comprises about 40% by weight of propylene glycol.

In a particularly preferred embodiment, the pharmaceutical composition of the invention comprises from about 5 to about 50% by weight of ethanol, more preferably from about 10 to about 30% weight of ethanol. In most preferred embodiments, the pharmaceutical composition of the invention comprises about 20% by weight of ethanol.

Several materials identified as carriers have also been found to be effective co-solubilizers, either alone or in combination with other viscosity-reducing agents, or certain other carriers. In general, any solvent in which paclitaxel or other taxanes are at least moderately soluble at body temperature or with gentle heating can be used as a co-solubilizer in the vehicle of the novel compositions. Preferred co-solubilizers are those in which at least 25 mg/ml of paclitaxel or other taxane can be dissolved at about 20-25° C. Some embodiments of the invention comprise more than one co-solubilizer. In some preferred embodiments, the compositions of the invention include at least two solubilizers.

The term “stabilizer” as used herein denotes a moiety that increases the stability of a taxane. Stabilizers according to the invention may stabilize taxanes by decreasing the rates of solvolysis (e.g., loss of the ester side chain at C-13 or deacetylation at C-10) and/or epimerization of the taxane molecule (e.g., at C-7) as compared to taxane. The stabilization of a taxane by a stabilizer according to the invention is detectable by a reduction of one or more known degradation products (e.g., 7-epi-taxol C, 10-deacetyltaxol, 7-epi-taxol, 7-epi-10-deacetyl-taxol, baccatin III, 10-deacetylbaccatin III, cephalomannine, nitine, 7-epi-cephalomannine (see, for example, Miller et al., J. Org. Chem. 46:1469-1474 (1981) and Volk, et al., J. Chromatography B 696: 99-115 (1997). In a particularly preferred embodiment of the invention, the stabilizer is ascorbic acid 6-palmitate (i.e., ascorbyl palmitate). Other stabilizers useful in the present invention include metal salts of acids such as alpha-hydroxy or beta-hydroxy acids, metal sulfates (e.g., FeSO₄, metal alpha-hydroxymethylsulfinates and metal sulfonates. The metal salts are the subject of Applicants' commonly owned U.S. patent application Ser. No. PCT/US01/09416 entitled “Uses of Metal Salts to Stabilize Taxane-based Compositions,” filed of even date herewith, and incorporated herein by reference.

Without wishing to be bound by any particular theory limiting the invention, Applicants believe that some stabilizers reduce taxane degradation by inhibiting the formation of radicals and/or by the formation of a complex between neighboring polar oxygen containing substituents in the taxane skeleton. This new configuration creates a “lock” which holds these chemical groups in place. Minimizing the interaction of these substituents with the surrounding medium therefore decreases the rates of solvolysis and/or deprotonation of those sites and thus decreases the rate of degradation of the parent compound. Hence, in some embodiments of the invention, preferred stabilizers are radical inhibitors. Radical inhibitors are well known in the art (see e.g., Remington's Pharmaceutical Sciences, supra)). Non-limiting representative radical inhibitors according to the invention include Fe²⁺ gluconate, Cu²⁺ gluconate, Zn²⁺ gluconate, Ca²⁺ ascorbate, HOCH₂SO₂Na, ascorbyl palmitate, beta-carotene, zinc methionine and zinc citrate.

Yet other preferred stabilizers contemplated by the inventors may additionally aid in preserving the color of the pharmaceutical compositions. An example of this type of stabilizer is dl-alpha-tocopherol, commercially available from BASF (Mt. Olive, N.J.).

The preferred range of the amount of stabilizer present in the compositions of the invention is from about 0.2% to about 1.0% by total weight of the composition. In general, the amount ranges from about 0.05% to about 2.0% by weight. Determinations are to whether a given substance functions as a stabilizer for purposes of the present invention, and if so, the optimal amount to add to the composition, are made by routine experimentation. For example, taxane formulations containing varying amounts of the compound are subjected to stress conditions (e.g., 80° C. for 24 hours) and then analyzed by HPLC. The formulations are compared to a control (not containing the compound) and the percentage of unchanged taxane is calculated from the HPLC profile. Compounds that achieve taxane ratios of 97% are generally considered acceptable; compounds achieving ratios greater than 98.5% are preferred. See also the Miller and Volk publications, above.

Pharmaceutical compositions according to the invention may include more than one type of carrier, co-solubilizer, or stabilizer. In some embodiments, the compositions of the invention may optionally be formulated with additional components, such as for example surfactants, pharmaceutical excipients, diluents, sweeteners, flavoring agents or coloring agents, as described in more details herein. Conventional pharmaceutical excipients, diluents, sweeteners, flavoring agents, coloring agents and any other inert ingredients regularly included in dosage forms intended for oral administration are well known in the art (see Remington's Pharmaceutical Sciences, supra).

A “surfactant” according to the invention is an amphiphilic moiety having a surface-active group capable of maintaining and/or promoting the dispersion of an hydrophobic compound within an aqueous media. One of skill in the art will appreciate that surfactants suitable in the compositions of the invention are well known in the art. Non-limiting representative surfactants include Vitamin E (e.g. alpha-tocopherol) and beta-carotene.

The term “taxane” is used to identify a diterpene moiety that is only slightly soluble in water. Taxanes according to the invention include without limitation moieties isolated from the Pacific yew tree (Taxus brevifolia) as well as derivatives, analogs, metabolites and prodrugs, and other taxanes. Preferably, the taxane is selected from the group consisting of paclitaxel, docetaxel, derivatives, analogs, metabolites and prodrugs of paclitaxel or docetaxel, and salts, polymorphs and hydrates thereof. More preferably, the taxane comprises paclitaxel. In some embodiments of the invention more than one taxane is included as active ingredient.

The taxane concentration in the compositions of the invention may vary based on the carrier(s) co-solubilizer(s) and/or stabilizers selected and on the desired total dose of taxane to be administered orally to the mammal. The concentration of taxane in the pharmaceutical compositions according to the invention may range from about 2 to about 100 mg/ml, preferably from about 6 to about 60 mg/ml or more, preferably from about 10 to about 50 mg/ml.

Applicants have discovered that the administration of an effective oral amount of a bioavailability-enhancing agent in conjunction with the administration of the compositions according to the invention furthers the achievement of a blood level of the taxane that is comparable to the blood level achieved by intravenous injection of the taxane. As discussed infra, a bioavailability-enhancing agent may be administered before, at the same time, or immediately after the administration of the compositions of the invention. Accordingly, in some preferred embodiments of the invention, the pharmaceutical compositions include a bioavailability-enhancing agent.

The term “bioavailability enhancing agent” also referred to as “enhancing agent” or “enabling agent”, is used to refer to an agent capable of promoting the absorption or bioavailability of another agent. Preferred bioavailability enhancing agents include cyclosporins and related oligopeptides produced by species in the genus Topycladium, ketoconazole, dexverapamil, amiodarone, nifedipine, reserpine, quinidine, nicardipine, ethacrynic acid, propafenone, reserpine, amiloride, ergot alkaloids, cefoperazone, tetracycline, chloroquine, fosfomycin, ivermectin, tamoxifen VX-710, VX-853, genistein and related isoflavonoids, calphostin, ceramides, morphine, morphine congeners, other opioids and opioid antagonists. Cyclosporins are a group of nonpolar cyclic oligopeptides (some of which have immunosuppressant activity) produced by the genus Topycladium, including, e.g., Topycladium inflatum gams (formerly designated as Trichoderma polysporum), Topycladium terricola and other fungi imperfecti. The major component, cyclosporin A (cyclosporine or CsA), has been identified along with several other analogs, for example, cyclosporins B through Z, some of which exhibit substantially less immunosuppressive activity than cyclosporin A. A number of synthetic and semi-synthetic analogs have also been prepared. See generally Jegorov et al., Phytochemistry, 38:403-407 (1995). The present invention comprehends natural, semi-synthetic, synthetic analogs, and derivatives of cyclosporins. Cyclosporins, particularly cyclosporine (cyclosporin A), are known inhibitors of the P-glycoprotein efflux pump and other transporter pumps as well as of certain P450 degradative enzymes, but to date no effective regimens for applying this property clinically have been developed to the point of clinical and commercial feasibility or regulatory approval.

Cyclosporins which may be used in preferred embodiments of the invention include, but are not limited to: cyclosporins A through Z but particularly cyclosporin A (cyclosporine), cyclosporin F, cyclosporin D, dihydro cyclosporin A, dihydro cyclosporin C, acetyl cyclosporin A, PSC-833, SDZ-NIM 811 which is (Me-Ile-4)-cyclosporin, an antiviral, non-immunosuppressive cyclosporin. Preferred cyclosporin compositions are described in WO 98/10747 and in WO 01/12229. Characteristic amino acid variations defining cyclosporins A-Z are described in Table 1 below.

TABLE 1
Cyclosporins A-Z

| Cy | Amino acids | | | | | | | | | | |
|---|---|---|---|---|---|---|---|---|---|---|---|
| CyA | Mebmt | Abu | Sar | MeLeu | Val | MeLeu | Ala | D-Ala | MeLeu | MeLeu | MeVal |
| CyB | Mebmt | Ala | Sar | MeLeu | Val | MeLeu | Ala | D-Ala | MeLeu | MeLeu | MeVal |
| CyC | Mebmt | Thr | Sar | MeLeu | Val | MeLeu | Ala | D-Ala | MeLeu | MeLeu | MeVal |
| CyD | Mebmt | Val | Sar | MeLeu | Val | MeLeu | Ala | D-Ala | MeLeu | MeLeu | MeVal |
| CyE | Mebmt | Abu | Sar | MeLeu | Val | MeLeu | Ala | D-Ala | MeLeu | MeLeu | Val |
| CyF | Desoxy-Mebmt | Abu | Sar | MeLeu | Val | MeLeu | Ala | D-Ala | MeLeu | MeLeu | MeVal |
| CyG | Mebmt | Nva | Sar | MeLeu | Val | MeLeu | Ala | D-Ala | MeLeu | MeLeu | MeVal |
| CyH | Mebmt | Abu | Sar | MeLeu | Val | MeLeu | Ala | D-Ala | MeLeu | MeLeu | D-Mev |
| CyI | Mebmt | Val | Sar | MeLeu | Val | MeLeu | Ala | D-Ala | MeLeu | Leu | MeVal |
| CyK | Desoxy-Mebmt | Val | Sar | MeLeu | Val | MeLeu | Ala | D-Ala | MeLeu | MeLeu | MeVal |
| CyL | Bmt | Abu | Sar | MeLeu | Val | MeLeu | Ala | D-Ala | MeLeu | MeLeu | MeVal |
| CyM | Mebmt | Nva | Sar | MeLeu | Val | MeLeu | Ala | D-Ala | MeLeu | MeLeu | MeVal |
| CyN | Mebmt | Nva | Sar | MeLeu | Val | MeLeu | Ala | D-Ala | MeLeu | Leu | MeVal |
| CyO | MeLeu | Nva | Sar | MeLeu | Val | MeLeu | Ala | D-Ala | MeLeu | MeLeu | MeVal |
| CyP | Bmt | Thr | Sar | MeLeu | Val | MeLeu | Ala | D-Ala | MeLeu | MeLeu | MeVal |
| CyQ | Mebmt | Abu | Sar | Val | Val | MeLeu | Ala | D-Ala | MeLeu | MeLeu | MeVal |
| CyR | Mebmt | Abu | Sar | MeLeu | Val | Leu | Ala | D-Ala | MeLeu | Leu | MeVal |
| CyS | Mebmt | Thr | Sar | Val | Val | MeLeu | Ala | D-Ala | MeLeu | MeLeu | MeVal |
| CyT | Mebmt | Abu | Sar | MeLeu | Val | MeLeu | Ala | D-Ala | MeLeu | Leu | MeVal |
| CyU | Mebmt | Abu | Sar | MeLeu | Val | Leu | Ala | D-Ala | MeLeu | MeLeu | MeVal |
| CyV | Mebmt | Abu | Sar | MeLeu | Val | MeLeu | Ala | D-Ala | MeLeu | MeLeu | MeVal |
| CyW | Mebmt | Thr | Sar | MeLeu | Val | MeLeu | Ala | D-Ala | MeLeu | MeLeu | Val |
| CyX | Mebmt | Nva | Sar | MeLeu | Val | MeLeu | Ala | D-Ala | Leu | MeLeu | MeVal |
| CyY | Mebmt | Nva | Sar | MeLeu | Val | Leu | Ala | D-Ala | MeLeu | MeLeu | MeVal |
| CyZ | MeAmino octyl | Abu | Sar | MeLeu | Val | MeLeu | Ala | D-Ala | MeLeu | MeLeu | MeVal |
| | Acid | | | | | | | | | | |

Cy = cyclosporin

In a more preferred embodiment, the invention provides a long term-stable pharmaceutical composition for oral administration to a mammal including a taxane, Vitamin E TPGS, propylene glycol, ethanol and ascorbyl palmitate.

A particularly preferred embodiment of the invention comprises the following ingredients:

| Ingredients | % w/v | U/mL | |
|---|---|---|---|
| Paclitaxel | 1.20 | 12.0 | mg |
| Vitamin E TPGS(*) | 40.00 | 400.00 | mg |
| Propylene glycol USP | 40.00 | 400.00 | mg |
| Ascorbyl Palmitate NF | 0.50 | 5.0 | mg |
| dl-alpha-tocopherol USP | 0.50 | 5.0 | mg |
| Dehydrated Alcohol | q.s. to 100 mL | q.s. to 1.0 | mL |

(*)d-alpha-tocopheryl polyethylene glycol 1000 succinate

Another particularly preferred embodiment of the invention comprises the following ingredients:

| Ingredient | Weight (grams) |
|---|---|
| Paclitaxel | 1.2 |
| Vitamin E TPGS (*) | 40.0 |
| Propylene glycol | 35.0 |
| Ascorbyl Palmitate | 0.50 |
| Vitamin E | 0.50 |
| Anhydrous Ethanol | ≈22.3 (28.1 mL) |

(*)d-alpha-tocopheryl polyethylene glycol 1000 succinate.

In these embodiments, the propylene glycol is present in an amount of from about 35 to about 40% of the weight of the composition.

The compositions of the invention may be prepared by any conventional method known to individuals of skill in the pharmaceutical arts for preparing liquid or other fluid oral formulations containing surfactant carriers and lipophilic active ingredients. Suitable non-limiting representative methods of preparing the compositions of the invention include for example the protocols described in examples herein. Since the majority of the preferred carriers are very viscous at room temperature, and in some cases retain a relatively high viscosity even upon the addition of a minor proportion of co-solubilizer, it is generally preferred in preparing the compositions to mix the carriers and co-solubilizers to be used, add the taxane active ingredient, and heat the resulting mixture while stirring, for example to about 40° C. This method enables the preparation of clear solutions. Certain co-solubilizers, however, particularly PHARMASOLVE™, lower the carrier viscosity and enhance taxane solubility to such a degree that the composition can be prepared by stirring at room temperature with no heating. It is desirable that the viscosity of the finished composition not be higher than 40,000 cps at body temperature (approximately 37° C.).

The oral compositions of the invention may be in the form of true solutions, emulsions or suspensions, but solutions of the active taxane ingredient in the carrier or carrier/co-solubilizer system are preferred.

The invention also sets for the methods of using the compositions for a variety of purposes including, but not limited to therapeutic applications. Thus, in a second aspect, the invention provides methods to achieve target blood level of taxane in a mammal by the oral administration of an effective amount of a pharmaceutical composition as described herein. Such methods are suitable to provide a target blood level of the taxane which is comparable to that achieved by intravenous administration of the taxane. Although some of the oral pharmaceutical compositions of the invention may provide target blood levels, including therapeutic blood levels of paclitaxel, when administered alone, a preferred method of the invention is to administer the oral pharmaceutical compositions concomitantly with the administration of at least one dose of an oral bioavailability enhancing agent because the levels of taxane that are subsequently achieved are in fact associated with pharmacological activity of the taxane.

Pharmaceutical compositions and bioavailability enhancing agents useful according to this aspect are as described for the first aspect of the invention.

“Target blood levels” according to the invention are blood concentrations of a taxane at or above the threshold concentrations necessary to observe the particular activities associated with taxanes that are sought. Non-limiting representative examples include the inhibition of tubulin disassembly, which occurs at blood levels of about 0.1 μM or about 85 ng/ml and the inhibition of protein isoprenylation (which occurs at blood levels of about 0.03 μM or about 25 ng/ml). Additionally, taxanes such as paclitaxel have been shown to inhibit angiogenesis and to inhibit the phosphorlation of intracellular Bcl-2. Some of these activities (such as the direct inhibition of oncogene functions or the inhibition of a transducing element) are directly related to taxane antitumorigenic properties. Hence, in some particularly preferred embodiments of the invention target blood levels are therapeutic blood levels at which a particular pharmacological activity is observed. Target blood levels may vary considerably due to a number of variables such as for example, use of concomitant medications, hepatitic status, albumin levels in the mammal being treated and variations between different pharmaceutical formulations. Target blood levels may be easily ascertained by routine methodologies such as the administration of the compositions of the invention in step-wise increments while monitoring paclitaxel concentration in the mammal.

In preferred embodiments of the invention wherein the mammal is a human in need of a regimen to inhibit of tubulin disassembly, target blood levels are at least about 0.1 μM or about 85 ng/ml for a period of time (e.g., several hours). In some embodiments of the invention wherein the mammal is a human in need of a regimen to inhibit protein isoprenylation, target blood levels are at least about 0.03 μM or about 25 ng/ml. Such target blood levels include without limitation, blood levels from about 25 ng/ml to about 85 ng/ml.

In a third aspect, the invention provides a method to investigate the physical properties of diterpenoids. More specifically, the invention provides tools useful to investigate the biochemical properties of taxane moieties in novel formulations capable of mediating larger increases in tissue distribution in vivo, without an increase in toxicity. Such tools, capable of expanding taxane volume of distribution, will allow investigators to elucidate a variety of biochemical properties in vivo, such as for example the effects of paclitaxel on the level of tubulin and/or microtubule-associated proteins (MAPs) overexpression, cell cycle progression, and nucleation of microtubule assembly in various tissues. Such studies promise to lead to a more comprehensive pharmacokinetic and pharmacological description of taxanes essential to identify novel applications and possibly to further optimize already existing therapeutic outcomes.

Finally, the methods and compositions according to the invention are useful in therapeutic approaches to taxane-responsive diseases. A “taxane-responsive disease” is used to refer to any condition including a disease condition, which is ameliorated by the oral administration of effective amounts of the pharmaceutical compositions described herein. Generally, a taxane responsive disease is characterized by uncontrolled cellular proliferation including, but not limited to the heterogeneous diseases of cancer, tumors, angiogenesis, psoriasis and polycystic kidney disease. As discussed supra, non-limiting representative examples of taxane-responsive diseases include cancers, tumors, Kaposi's sarcoma, malignancies, uncontrolled tissue and cellular proliferation secondary to tissue injury. Among the types of carcinoma that may be treated particularly effectively according to the methods of the invention, are hepatocellular carcinoma and liver metastases, cancers of the gastrointestinal tract, pancreas, prostate and lung, and Kaposi's sarcoma. Non-cancerous diseases that may be effectively treated in accordance with the present invention are uncontrolled tissue or cellular proliferation secondary to tissue injury, polycystic kidney disease, inflammatory diseases (e.g., arthritis) and malaria, including chloroquine- and pyrimethamine-resistant malaria parasites (Pouvelle, et al., supra).

The terms “treatment” or “treating” as used herein with reference to a taxane responsive disease refer to prophylaxis and to the amelioration of symptoms already present in an individual by altering the taxane blood levels. It will be appreciated by a person of skill that a treatment need not be completely effective in preventing the onset of a disease or eliminating the symptoms associated with a disease, nor does a treatment need to cure a disease in order to be effective. Any reduction in the severity of the symptoms, delay in the onset of symptoms, or delay in the rate of progression of severity of symptoms is contemplated. Persons at risk of developing a taxane-responsive disease may be treated prophylactically based on any variety of factors suggesting the possible onset of the disease, e.g., family history, environmental exposure, genetic markers, early symptoms, and the like.

As discussed for other aspects, although some of the oral pharmaceutical compositions of the invention may provide target blood levels, including therapeutic blood levels, of the taxane when administered alone, the preferred method of the invention for treating a mammal suffering from taxane-responsive disease is to administer the oral compositions containing a taxane such as paclitaxel concomitantly with the administration of an oral bioavailability enhancing agent. Hence, a preferred embodiment of the method of the invention comprises the oral administration an enhancing agent simultaneously with, or prior to, or both simultaneously with and prior to the oral administration to increase the quantity of absorption of the taxane into the bloodstream. Pharmaceutical compositions and bioavailability enhancing agents useful according to this aspect of the invention are as described for the first aspect of the invention.

In general, the dosage range of the bioavailability enhancing agent to be co-administered with the taxane in accordance with the invention is from about 0.1 to about 20 mg/kg of patient body weight, preferably from about 3 to about 15 mg/kg of patient body weight, and more preferably from 5-10 mg/kg. “Co-administration” of the enhancing agent comprehends administration substantially simultaneously with the taxane (either less than 0.5 hr. before, less than 0.5 hr. after or together), from about 0.5 to about 72 hr. before the administration of the taxane, or both, i.e., with one or more doses of the same or different enhancing agents given at least 0.5 hr. before and one dose given substantially simultaneously with (either together with or immediately before of after) the target agent. Additionally, “co-administration” comprehends administering more than one dose of taxane within 72 hr. after a dose of enhancing agent, in other words, the enhancing agent(s) need not be administered again before or with every administration of taxane, but may be administered intermittently during the course of treatment.

“Effective amounts” is used to denote known amounts of the taxane in the pharmaceutical compositions of the invention sufficient to achieve a particular taxane blood level. The dosage range of the orally administered taxane in the compositions of the invention will vary in accordance with a number of factors, including the particular taxane, on its therapeutic index, the requirements of the disease being treated, the age and condition of the mammal, the nature of the disease(s) being treated the stage of the disease, other medications and being taken by the mammal, and the like. The pharmacology and pharmacokinetics of taxanes, especially paclitaxel and docetaxel, are well known. This pharmacological information can be used in conjunction with the exigencies of the mammal being treated to optimize dosing and scheduling regimens. One of skill in the art will appreciate that specific dosing and scheduling of this composition may be tailored to meet the requirements of each patient by trial and error while monitoring the patient's response (see Rowinsky, Oncology 11(3):7-19 (1997) for dosing and scheduling considerations).

Precise amounts of each of the taxane included in the oral dosage forms will vary depending on the age, weight, disease and condition of the patient. For example, paclitaxel or other taxane dosage forms may contain sufficient quantities of the target agent to provide a daily dosage of about 20-200 mg/m² (based on the mammal/patient body surface area) or about 0.5-30 mg/kg (based on mammal/patient body weight) as single or divided (2-3) daily doses. Preferred dosage amounts are about 50-200 mg/m² or about 2-6 mg/kg to maintain blood levels of taxane in the range of 50-500 ng/ml for extended periods of time (e.g., 8-12 hours) after each oral dose. These levels are at least comparable to those achieved with 96-hour IV infusion paclitaxel therapy (which unlike oral administration causes the patient great inconvenience, discomfort, loss of quality time, infection potential, etc.) (Wilson et al., J. Clin. Oncol. 12:1621-1629 (1994)). Moreover, such blood levels of paclitaxel are more than sufficient to provide the desired pharmacological activities of the target drug, e.g., inhibition of tubulin disassembly and inhibition of protein isoprenylation which are directly related to its antitumor effects by inhibiting oncogene functions and inhibition of signal-transducing proteins postulated to play a pivotal role in cell growth regulation.

Preferred dosing schedules for administration of oral paclitaxel are (a) the daily administration to a patient in need thereof of 1-3 equally divided doses providing about 20-1000 mg/m² (based on body surface area), and preferably about 50-200 mg/m², with daily administration being continued for 1-4 consecutive days each 2-3 weeks, (b) administration for about one day each week, and (c) daily administration for two or three weeks, followed by a one week rest period. The former schedule is comparable to use of a 96-hour paclitaxel infusion every 2-3 weeks, which is considered by some a preferred IV treatment regimen.

In a particularly preferred embodiment of the invention, the pharmaceutical composition administered comprises about 60 mg/m² paclitaxel by weight. In another particularly preferred embodiment, the pharmaceutical composition comprises about 180 mg/m² by weight.

Two or more different enhancing agents and/or two or more different taxane target agents may be administered together, alternately or intermittently in all of the various aspects of the method of the invention.

As discussed infra, oral paclitaxel administered alone (e.g., in a solid dosage form or even in a liquid vehicle not containing an oral absorption promoting carrier) exhibits near zero bioavailability. Upon oral administration of the compositions of the inventions one hour after administration of an effective oral dose of an oral bioavailability enhancing agent, the amount of the taxane absorbed into the bloodstream is at least about 15% of the amount absorbed when the same dose of paclitaxel is administered to intravenously in a standard intravenous vehicle e.g., example a CREMOPHOR™ EL/ethanol vehicle. The relative percentage of absorption is determined by standard methodologies in the field such as by comparing the respective AUC (is the area under the plasma concentration-time curve, commonly used in pharmacokinetics to quantify the percentage of drug absorption and elimination determined after oral/intravenous administration of the drug. A high AUC is an indication that the drug tested is more likely to be available to reach the target tissue or organ. The novel pharmaceutical compositions may be administered in any known pharmaceutical dosage form. For example, the compositions may be encapsulated in a soft or hard gelatin capsule or may be administered in the form of a liquid preparation.

Oral administration of taxanes in accordance with the invention may actually decrease toxic side effects in many cases as compared with currently utilized IV therapy. Rather than producing a sudden and rapid high concentration in blood level as is usually the case with an IV infusion, absorption of the active agent through the gut wall (promoted by the enhancing agents), provides a more gradual appearance in the blood levels. A stable, steady-state maintenance of those levels at or close to the ideal range for a long period of time can be more easily achieved with oral administration than with the inconvenience and risk of infection in an already immuno-compromised host.

In a further embodiment of the present invention, the oral compositions of the invention may be administered in a two-part medicament system (e.g., to accommodate the use of carriers which are chemically or physically incompatible with desired adjunctive ingredients such as flavoring or coloring agents). In such cases, the taxane may be administered to the patient as the first part of the medicament in a solubilizing vehicle, which may be sweetened, flavored or colored as desired. The administration of the taxane may be followed by administration of a larger volume of fluid, for example 1 to 8 fluid ounces (30-240 ml), containing at least one carrier or a carrier/co-solubilizer system in accordance with the invention. It has been discovered that administration of the second, “chaser” formulation a short time after the taxane can retard precipitation of the taxane which might otherwise occur upon entry into the gastric fluid and promote oral absorption to a degree comparable to that observed when the taxane is intermixed with the carrier and administered simultaneously.

Illustrative examples of “chaser” formulations that may be used in a two-part oral taxane medicament include:

a) 2-20% (by weight) Vitamin E TPGS+water q.s.;

b) 2- 25% Vitamin E TPGS+2-25% PHARMASOLVE™+water q.s.; and

c) 2- 20% Vitamin E TPGS+2-25% propylene glycol+water q.s.

Pursuant to yet another aspect of the invention, the oral compositions of the invention contain not only one or more taxane but also one or more bioavailability enhancing agents in a combination dosage form. For example, such combination dosage form may contain from about 0.1 to about 20 mg/kg (based on average patient body weight) of one or more of cyclosporins A, D, C, F and G, dihydro CsA, dihydro CsC and acetyl CsA together with about 20 to about 1000 mg/m² (based on average patient body surface area), and preferably about 50-200 mg/m² of paclitaxel, docetaxel, other taxanes or paclitaxel or docetaxel derivatives.

The compositions and methods of the present invention provide many advantages in comparison with prior art and intravenous regimens (e.g., added stability, overall palatability, decreased toxicity due to lower peak levels, patient convenience and comfort, ease of administration and lowered expense). In addition, the compositions and methods of the invention greatly reduce the likelihood of allergic hypersensitivity reactions common with IV administration, thereby reducing or overcoming the need for pre-medication regimens (such as H-1and H-2 blockers plus steroids). The latter is of particular relevance in the treatment of diabetic cancer patients since it is known that steroids may cause diabetes mellitus.

The present invention provides for the administration of taxanes, e.g., paclitaxel, in comparatively infrequent daily doses (e.g., about twice/day) and/or according to schedules that would otherwise not be possible or practical with the intravenous route. The once-a-day administration of a bioavailability enhancer (e.g., cyclosporin A) may suffice even if more than one dose of taxane is administered during the day. Hence, for example, paclitaxel could be given intermittently as single dose on a fixed schedule (weekly, biweekly, etc.) or chronically, over a period of consecutive days (e.g., 4 days) every 2-4 weeks with the goal of keeping the levels within a safe and effective “window”.

The following examples are intended to further illustrate certain preferred embodiments of the invention and are not limiting in nature. These examples are not intended, however, to limit the invention in any way or to set forth specific active ingredients, carriers, co-solubilizers, enhancer agents, dosage ranges, testing procedures or other parameters that must be used exclusively to practice the invention. Hence, the use of paclitaxel to illustrate aspects of taxanes as a whole is purely for illustrative purposes and should not be construed as limiting the invention.

Those skilled in the art will recognize, or be able to ascertain, using no more than routine experimentation, numerous equivalents to the specific substances and procedures described herein. Such equivalents are considered to be within the scope of this invention, and are covered by the following claims.

### EXAMPLE 1

### Preparation of Representative Pharmaceutical Compositions

One of skill in the field will readily appreciate that a variety of protocols may be used to prepare representative compositions according to the invention. The following is included merely to illustrate the ease with which representative compositions according to the invention may be prepared. Representative formulations designated as Formulas I and IA were prepared.

Formula I

| Ingredients | % w/v | U/ml | |
|---|---|---|---|
| Paclitaxel | 1.20 | 12.0 | mg |
| Vitamin E TPGS(*) | 40.00 | 400.00 | mg |
| Propylene glycol USP | 40.00 | 400.00 | mg |
| Ascorbyl Palmitate NF | 0.50 | 5.0 | mg |
| dl-alpha-tocopherol USP | 0.50 | 5.0 | mg |
| Dehydrated Alcohol | q.s. to 100 ml | q.s. to 1.0 | ml |

Formula IA

| Ingredient | Weight (grams) |
|---|---|
| Paclitaxel | 1.2 |
| Vitamin E TPGS | 40.0 |
| Propylene glycol | 35.0 |
| Ascorbyl Palmitate | 0.5 |
| Vitamin E | 0.5 |
| Anhydrous Ethanol | −22.3 (28.1 mL) |

(*)d-alpha-tocopheryl polyethylene glycol 1000 succinate

Paclitaxel (NaPro BioTherapeutics, Inc., Boulder, Colo.), Ascorbyl Palmitate NF (Aldrich Chemical Co., Milwaukee Wis.), and dl-alpha-tocopherol USP (Roche Vitamins, Nutley, N.J.) in the amounts specified above were placed in a suitable volumetric container and dispersed in at least two-thirds of the total amount of dehydrated ethanol (Florida Distillers Co., Lake Alfred, Fla.) to be included (either 1.0 or 100 ml). Upon complete dispersion, the appropriate amount of propylene glycol was added and mixed for at least 30 minutes. Liquefied Vitamin E TPGS (d-alpha-tocopheryl polyethylene glycol 1000 succinate (Eastman Chemical Co., Kingsport, Tenn.) (by heating it separately to approximately 50-60° C. or until it liquefies) was added. The remainder of the dehydrated alcohol was then added and the final formulation was cooled slowly to approximately 25-30° C. (room temperature). Once the solution reached room temperature, the solution was adjusted to the final volume with ethanol while stirring constantly until a light yellow transparent solution was formed.

### EXAMPLE 2

### Stability Analysis

As discussed supra, one of the advantages of the compositions of the invention is their stability. The following experiment illustrates the stability of the compositions according to the invention. The representative compositions prepared as described in Example 1 were assayed in compliance with ICH guidelines. Using a suitable size Eppendorf Pipette, 10.2-10.5 ml of solution was delivered into individual 15 cc amber glass bottles using a 28/400 Black Phenolic Cap with Poly Seal Liner. Gross, tare and net weight of each bottle were recorded. The bottles were then placed upright at 40° C. and 75% humidity. Subsets of bottles were removed and tested according to methodology well known in the field (i.e., presence of known degradation products by HPLC after each time point as shown below (2 weeks, and 1-6 months). As shown in Table 2 below, the compositions were found to be stable showing minimal levels (expressed as a % of total impurities) of compounds considered hallmarks of paclitaxel degradation such as 7-epi-Taxol C, 10-deacetyltaxol, or baccatin III as compared to negative control formulations based on CREMOPHORE EL™ (data not shown). In addition, impurities were less than 3.5% after as long as six months of incubation (data not shown).

TABLE 2
Stability Analysis

| DEGRADATION | | 2 | 1 | 2 | 3 | 6 |
|---|---|---|---|---|---|---|
| PRODUCTS | INITIAL | WEEKS | MONTH | MONTHS | MONTHS | MONTHS |
| 7-Epi-Taxol C | ND | ND | ND | ND | ND | 0.11 |
| 10-Deacetyltaxol | ND | 0.04 | 0.04 | 0.11 | 0.12 | 0.04 |
| 7-Epi-taxol | 0.06 | 0.07 | 0.06 | 0.06 | 0.06 | 0.16 |
| 7-Epi-10-deacetyl-taxol | 0.14 | 0.15 | 0.17 | 0.15 | 0.14 | 0.21 |
| Baccatin III | ND | 0.07 | 0.09 | 0.13 | 0.14 | 0.18 |
| 10-deacetylbaccatin III | ND | 0.02 | 0.02 | 0.04 | 0.02 | 0.02 |
| 7-Epi-Cephalomannine | ND | ND | ND | ND | ND | 0.05 |

### EXAMPLE 3

### Solubility Analysis

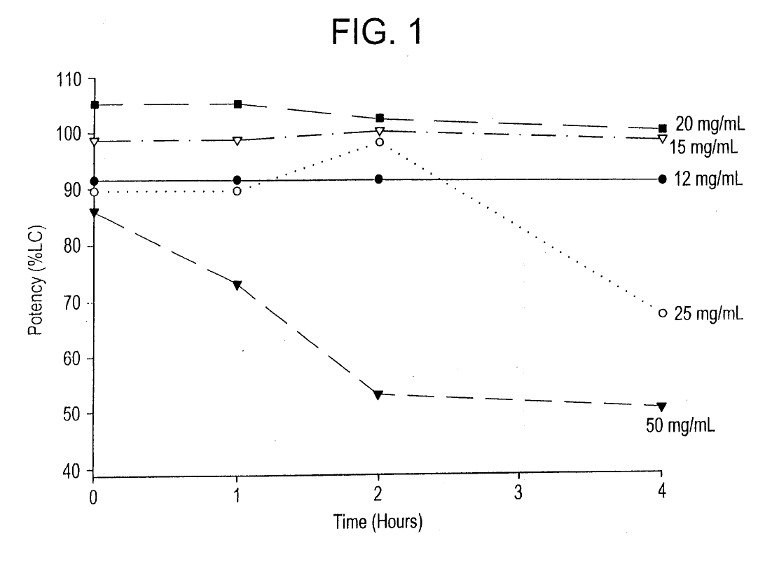

To assess paclitaxel solubility in representative compositions of the invention, formulations prepared as per Formula I, but having final paclitaxel concentrations of 12, 15, 25, and 50 mg/ml were diluted with water to a 1 to 11 ratio (1 ml paclitaxel formulation and 10 ml water). The solutions were then assayed by HPLC analytical method. As shown in FIG. 1, paclitaxel remained in solution for at least two hours (thus showing solubility for an adequate period of time) in all preparations with the exception of the 50 mg/ml preparation. Notably, preparations containing between 12 to 20 mg/ml remained in solution for the entire duration of the study.

### EXAMPLE 4

### Pharmacokinetic Analysis

The compositions and methods of the invention are used to achieve target blood levels, including therapeutic blood levels, of taxane in a mammal. To exemplify this aspect of the invention, two groups of patients (total of five patients) were first administered an enhancing agent preparation such as Neoral® 5 mg/kg (Cyclosporin A, Novartis Pharmaceuticals, Inc., Summit, N.J.) and 30 minutes later were administered Formula 1 at single doses of 60 mg/m² (n=2) and 180 mg/m² (n=3) of paclitaxel. Serial blood samples were taken frequently over 30-48 hours and assayed for paclitaxel. Individual and mean pharmacokinetic parameters of paclitaxel are shown in Table 3. These results show slightly higher values for Cₘₐₓ and AUC following formula one than for the CREMOPHOR EL™ formulation. With both doses therapeutic blood levels were achieved and there was an approximate 2-fold increase in systemic exposure of paclitaxel when one compares the area under the plasma concentration vs. time curve for the 2 doses. The latter suggests that the compositions of the invention may provide sufficient levels of paclitaxel in plasma with ingestion of less ethanol than the CREMOPHOR EL™ based formulations.

TABLE 3
Comparison of Pharmacokinetic Parameters

| | | | AUC_{inf} |
|---|---|---|---|
| PATIENT | Cₘₐₓ (ng/ml) | AUC last (ng · hr/ml) | (ng · hr/ml) |
| 1 | 189.3 | 929 | 1025 |
| 2 | 226.6 | 1126 | 1208 |
| Mean | 207.9 | 1029 | 1159.5 |
| SD | 26.3 | 137.2 | 146.4 |
| CV | 19.6 | 22.3 | 21.2 |

Table 4 shows a comparison of the pharmacokinctic parameters for a CREMOPHOR EL™ (a polyethoxylated castor oil )/EtOH based formulation (n=9) versus those for Formula One (n=4) according to the invention.

TABLE 4
Comparison of Pharmacokinetic Parameters (C/E vs. Formula One)

| | | FORMULA ONE |
|---|---|---|
| | | (AUC_{IV} = 50% |
| | C/E | of CE value) |
| APPARENT BIOAVAILABILITY @ | 42% | 69.1% |
| 60 MG/m² | | |
| AUC∞- 60 mg/m² | 1409 (56) | 1159.5 |
| AUC∞- 180 mg/m² | 2844 (70) | 2474 |
| AUC RATIO | 2.0 | 2.1 |

### EXAMPLE 5

### Palatability Test

Another property of the compositions of the invention is their palatability as compared with their counterpart CREMOPHOR™ EL (a polyethoxylated castor oil)/EtOH based formulations. Formulations prepared with traditional stabilizers have an unpleasant bitter taste probably due to the castor oil. For this purpose 5 ml aliquots of Formula I (40% Vitamin E. TPGS+40% propylene glycol+20% ethanol, see Example 1) and 75% CREMOPHOR™ EL+25% Ethanol were placed in 17 glass vials (an additional vial without formulations was used as a negative control). Various flavors commercially available from International Flavor & Fragrances, Inc., Dayton, N.J.; Crompton & Knowles, Charlotte, N.C. and Virginia Dave, Brooklyn, N.Y.) as shown in Table 5 were added to 16 of these vials as follows: banana (0.5%), cherry (0.2 and 0.5%), grape (0.5%), grape maskant (0.5%), mint (0.2 and 0.5%), peppermint (0.2 and 0.5%), herbal mint (0.2 and 0.5%), pharmasweet (0.1%), prosweet (1%), rainbow sorbet (0.5%), watermelon (0.5%), and wintergreen (0.5%). Preparations were administered blindly to test individuals to taste and score as either (−) no good; (+) acceptable/ok; (++) good; or (+++) excellent. The numbers were marked on the cap of two groups of sample vials that contained placebo (formula I or 75% Cremophor EL/25% ethanol with different flavor). Random solutions were taken from these vials by dropper. They were tasted by two chemists. The results versus number were recorded.

As shown in Table 5, Formula I in various preparations was found to be more palatable than counterpart formulations. Moreover, the banana-flavored preparation was found to be excellent.

TABLE 5
Flavor Testing

| | | (75% Cremophor |
|---|---|---|
| | FORMULA I | EL + 25% Ethanol |
| Blank* | +** | − |
| Banana (0.5%) | +++ | + |
| Cherry (0.2%) | ++ | + |
| Cherry (.5%) | ++ | + |
| Grape (0.5%) | ++ | + |
| Grape Maskant Flavor (0.5%) | ++ | + |
| Mint (0.2%) | ++ | + |
| Mint (0.5%) | ++ | + |
| Peppermint (0.2%) | ++ | + |
| Peppermint (0.5%) | ++ | + |
| Herbal Mint Flavor (0.2%) | ++ | + |
| Herbal Mint Flavor (0.5%) | ++ | + |
| Pharmasweet Flavor (0.1%) | ++ | + |
| Prosweet (1%) | ++ | + |
| Rainbow Sherbet (0.5%) | ++ | + |
| Watermelon (0.5%) | ++ | + |
| Wintergreen Flavor (0.5%) | ++ | + |

*No flavor was added;
Next column: **, −, no good, +, acceptable/ok, ++, good, +++, excellent.

### EXAMPLE 6

### Comparative Absorption Assays

The purpose of the following experiment was to illustrate the ability of representative compositions and methods of the invention to yield absorption values greater than those observed with prior art IV methodologies. For this purpose groups of three male rats each were fasted for 16-18 hours prior to dosing with ³H-radiolabeled paclitaxel. Each group of animals received one oral dose of cyclosporin A (5 mg/kg) prior to dosing with a representative pharmaceutical composition according to the invention including paclitaxel. One hour subsequent to cyclosporin dosing, each group received approximately 9 mg/kg of paclitaxel orally in a composition according to the invention. Each group received a different oral formulation. Blood samples were collected from each animal at 0.5, 1, 2, 3, 4, 6, 8, 12 and 24 hours post-dose of paclitaxel. The blood samples were combusted and assayed for total radioactivity. The total blood radioactivity levels (corresponding to concentration in the blood of ³H-paclitaxel) were plotted on a graph vs. time post-dose. Data for each group of rats were compiled in the form of mean AUC, Cₘₐₓ and Tₘₐₓ. The percentage of absorption of ³H-paclitaxel for each group of animals was calculated by comparing the mean AUC value for the group to the corresponding mean AUC of a reference group of rats administered 3H-paclitaxel (9 mg/kg) intravenously in the form of PAXENE™ (Baker Norton Pharmaceuticals, Miami Fla.) which includes CREMOPHOR™ EL, ethanol and citric acid. As shown in Table 6 several carriers and carrier/co-solubilizer combinations formulated into oral compositions containing paclitaxel in accordance with the invention were found to yield percentage absorption values in the experimental animals of 15% or greater in comparison with IV paclitaxel (data not shown).

TABLE 6
Carriers and carrier/co-solubilizer combinations which
achieved greater than 15% paclitaxel absorption

| CARRIERS | CO-SOLUBILIZERS | | | | | | |
|---|---|---|---|---|---|---|---|
| TPGS | Pharmasolve | Propylene | Mygliols | Softigen | PEG 200 | Propylene | PEG |
| | | glycol | | | & 400 | glycol/ | 200 & |
| | | | | | | Pharmasolve | 400/ |
| | | | | | | | Pharmasolve |
| Gelucire | Pharmasolve | Mygliols | Olive oil/ | Olive oil/ | Olive oil/ | Cremophor | Cremophor |
| 44/14 | | | Brij 97 | Cremophor | TPGS | EL | RH 40 |
| | | | | RH 40 | | | |
| Gelucire | Labrasol | TPGS/ | Tween 80 | PEG 400 | | | |
| 44/14 | | Solutol HS 15 | | | | | |
| Gelucire | Tween 80 | PEG 400 | Cremophor | | | | |
| 50/13 | | | EL | | | | |
| Cremophor | Pharmasolve | Citrate esters | EtOH/H₂O | EtOH | | | |
| EL | | | | | | | |
| Cremophor | EtOH/ | | | | | | |
| RH 40 | H₂O | | | | | | |
| Myrj 49 | Pharmasolve | | | | | | |
| Myrj 52 | Pharmasolve | Propylene | | | | | |
| | | glycol | | | | | |
| Myrj 53 | Pharmasolve | | | | | | |
| Tween 40* | | | | | | | |
| Tween 60* | | | | | | | |
| Tween 80* | EtOH | Citrate esters | Olive oil | PEG 400 | H₂O | | |
| Crillet 6* | | | | | | | |
| Emsorb | Pharmasolve | | | | | | |
| 2726 | | | | | | | |
| Solutol HS | | | | | | | |
| 15* | | | | | | | |
| Brij 76 | Pharmasolve | | | | | | |
| Brij 78 | Pharmasolve | | | | | | |
| Brij 98 | Pharmasolve | | | | | | |
| Crovol A- | | | | | | | |
| 40* | | | | | | | |
| Crovol M- | | | | | | | |
| 40* | | | | | | | |
| -Cyclo- | H₂O | | | | | | |
| dextrin | | | | | | | |

*Have been demonstrated to work as both solubilizer and carrier
Note:
All carriers listed above can solubilize paclitaxel greater than 25 mg/ml at 37° C.

### EXAMPLE 7

### Evaluation of Carriers

The experiments described hereinafter illustrate the ability of representative oral compositions formulated with different moieties as carriers to yield higher absorption rates than their respective IV counterparts when administered orally. Polyoxyethylated (POE) Sorbitan Fatty Acid Esters as Carriers

Table 7 lists formulations including certain POE sorbitan fatty acid esters as carriers for oral paclitaxel, alone or in combination with a co-solubilizer. In formulations where more than one component is present, the respective weight ratios of the components are given. Each of these formulations was tested in the animal model described supra and found to yield a percentage absorption of paclitaxel upon oral administration greater than 15% paclitaxel absorption. The table sets forth the total dose of paclitaxel incorporated into each vehicle as actually administered to the experimental animals, the concentration of paclitaxel in the composition, the HLB value of the carrier, the mean AUC value for the group of rats receiving the formulation and the percentage of paclitaxel absorption in comparison with rats receiving IV administration.

TABLE 7
Absorption Results of Polyoxyethylated (POE)
Sorbitan Fatty Acid Esters Surfactants as Carriers

| | Dose | Conc. | | AUC | % |
|---|---|---|---|---|---|
| FORMULATIONS | [mg/kg] | [mg/ml] | HLB | μg · eqxhr/ml | ABS* |
| POE 20 sorbitan monolaurate (Tween 20) | 10.2 | 18 | 16.7 | 17.2 | 54.6 |
| POE 20 sorbitan monopalmitate (Tween 40) | 10.2 | 18 | 15.6 | 17.6 | 55.9 |
| POE 20 sorbitan monostearate (Tween 60) | 8.9 | 25 | 14.9 | 17.1 | 62.3 |
| POE 20 sorbitan tristearate (Tween 65) | 9.4 | 25 | 10.5 | 6.15 | 21.1 |
| POE 20 sorbitan monooleate (Tween 80) | 9.0 | 18 | 15.0 | 11.4 | 40.9 |
| POE 20 sorbitan monoisostearate (Crillet 6) | 9.3 | 20 | 14.9 | 13.6 | 47.5 |
| POE 40 sorbitan diisostearate/Pharmasolve (3:1) | 10.2 | 25 | 15.0* | 7.76 | 24.6 |
| [Emsorb 2726] | | | | | |

*Percent absorption versus paclitaxel IV AUC (same for Tables 4-11)

POE Alkyl Ethers as Carriers

Table 8 summarizes data for formulations containing POE alkyl ethers as carriers. The data correspond to the data described in the preceding table.

TABLE 8
Absorption Results of Polyoxyethylated (POE)
Alkyl Ethers Surfactants as Carriers

| | Dose | Conc. | | AUC | % |
|---|---|---|---|---|---|
| FORMULATIONS | [mg/kg] | [mg/ml] | HLB | μg · eqxhr/ml | ABS |
| POE 20 stearate ester/ | 9.2 | 25 | 15.0* | 10.3 | 36.4 |
| Pharmasolve (3:1) | | | | | |
| [Myrj 49] | | | | | |
| POE 40 stearate ester/ | 9.4 | 18 | 16.9* | 16.2 | 57.3 |
| Pharmasolve (3:1) | | | | | |
| [Myrj 52] | | | | | |
| POE 50 stearate ester/ | 10.0 | 25 | 17.9* | 7.01 | 22.3 |
| Pharmasolve (3:1) | | | | | |
| [Myrj 53] | | | | | |

*Not an actual HLB value of mixture. Numbers represent HLB values of pure surfactants POE Stearates as Carriers

Table 9 summarizes data for formulations containing POE stearates as carriers. The data set forth correspond to the data described in Example 7.

TABLE 9
Absorption Results of Polyoxyethylated (POE)
Stearates as Carriers

| | Dose | Conc. | | AUC | % |
|---|---|---|---|---|---|
| FORMULATIONS | [mg/kg] | [mg/ml] | HLB | μg · eqxhr/ml | ABS |
| POE 20 stearate ester/ | 9.2 | 25 | 15.0* | 10.3 | 36.4 |
| Pharmasolve (3:1) | | | | | |
| [Myrj 49] | | | | | |
| POE 40 stearate ester/ | 9.4 | 18 | 16.9* | 16.2 | 57.3 |
| Pharmasolve (3:1) | | | | | |
| [Myrj 52] | | | | | |
| POE 50 stearate ester/ | 10.0 | 25 | 17.9* | 7.01 | 22.3 |
| Pharmasolve (3:1) | | | | | |
| [Myrj 53] | | | | | |

*Not an actual HLB value of mixture. Numbers represent HLB values of pure surfactants Ethoxylated Modified Triglycerides as Carriers

Table 10 summarizes data for formulations containing ethoxylated-modified triglycerides as carriers. The data set forth correspond to the data described in Example 7.

TABLE 10
Absorption Results of Ethoxylated
Modified Triglycerides as Carriers

| | Dose | Conc. | | AUC μg · | |
|---|---|---|---|---|---|
| FORMULATIONS | [mg/kg] | [mg/ml] | HLB | eqxhr/ml | % ABS |
| PEG-20 Almond | 9.5 | 20 | 10 | 8.06 | 27.6 |
| Glycerides (Crovol A-40) | | | | | |
| PEG-20 Corn Glycerides | 9.6 | 20 | 10 | 7.46 | 25.3 |
| (Crovol M-40) | | | | | |

POE 660 Hydroxystearates as Carriers

Table 11 summarizes data for formulations containing POE 660 hydroxystearates as carriers. The data set forth correspond to the data described in Example 7.

TABLE 11
Absorption Results of Polyoxyethylated (POE)
660 Hydroxystearate as Carriers

| | Dose | Conc. | | AUC μg · | |
|---|---|---|---|---|---|
| FORMULATIONS | [mg/kg] | [mg/ml] | HLB | eqxhr/ml | % ABS |
| POE 660 hydroxystearate | 9.1 | 25 | ˜14 | 10.8 | 38.4 |
| (Solutol HS 15) | | | | | |
| Gelucire 44/14 + Solutol | 9.3 | 25 | ˜14 | 6.54 | 22.8 |
| HS + TPGS (2:1:1) | | | | | |

Saturated Polyglycolized Glycerides as Carriers

Table 12 summarizes data for formulations containing saturated polyglycolized glycerides as carriers. The data set forth correspond to the data described in Example 7.

TABLE 12
Absorption Results of Saturated Polyglycolized Glycerides
as Carriers

| | | | | |
|---|---|---|---|---|
| | | | AUC | |
| | Dose | Conc. | μg · eqxhr/ | % |
| FORMULATIONS | [mg/kg] | [mg/ml] | ml | ABS |

| | | | | |
|---|---|---|---|---|
| Gelucire 44/14 + PEG 400 (6:1) | 10.3 | 25 | 11.9 | 37.4 |
| Gelucire 44/14 + Labrasol (6:1) | 9.3 | 25 | 12.1 | 42.1 |
| Gelucire 44/14 + Mygliol 810 | 8.7 | 25 | 4.75 | 17.6 |
| (6:1) | | | | |
| Gelucire 44/14 + Mygliol 818 | 10.3 | 25 | 8.45 | 26.6 |
| (6:1) | | | | |
| Gelucire 44/14 + Mygliol 840 | 9.5 | 25 | 6.48 | 22.0 |
| (6:1) | | | | |
| Gelucire 44/14 + Cremophore | 9.5 | 25 | 10.7 | 36.6 |
| RH 40 (6:1) | | | | |
| Gelucire 44/14 + Cremophor | 9.8 | 25 | 11.5 | 38.1 |
| EL (6:1) | | | | |
| Gelucire 44/14 + Solutol HS + | 9.3 | 25 | 6.54 | 22.8 |
| TPGS (2:1:1) | | | | |
| Gelucire 44/14 + Olive Oil + | 9.6 | 20 | 11.9 | 39.9 |
| Tween 80 (2:1:1) | | | | |
| Gelucire 44/14 + Olive Oil + | 9.6 | 20 | 9.83 | 33.2 |
| TPGS (2:1:1) | | | | |
| Gelucire 44/14 + Olive Oil + | 9.6 | 20 | 9.07 | 30.6 |
| POE 10 Oleyl (2:1:1) | | | | |
| Gelucire 44/14 + Olive Oil + | 9.1 | 20 | 7.73 | 27.5 |
| Cremophor RH 40 (2:1:1) | | | | |
| Gelucire 44/14 + Tween 80 (6:1) | 9.7 | 25 | 10.05 | 33.5 |
| Gelucire 50/13 + Tween 80 (5:2) | 9.4 | 25 | 8.21 | 28.4 |
| Gelucire 50/13 + PEG 400 (6:1) | 9.3 | 25 | 6.46 | 22.5 |
| Gelucire 50/13 + Cremophor EL | 9.1 | 25 | 8.11 | 28.9 |
| (6:1) | | | | |

Labrasol: Saturated polyglycolyzed C8-C10 glycerides (HLB = 14)
Mygliols: Neutral oils (saturated coconut and palm kernel fatty acids) mainly C8-n C10 fatty acids
Cremophor EL: Polyoxyl 35 castor oil (HLB 12-14)
Cremophor RH 40: Polyoxyl 40 Hydrogenated castor oil (HLB 14-16)

Vitamin E TPGS Systems as Carriers

Table 13 summarizes data for formulations containing Vitamin E TPGS systems as carriers. The data set forth correspond to the data described in Example 7.

TABLE 13
Absorption Results of TPGS Systems as Carriers

| | | | AUC | |
|---|---|---|---|---|
| | Dose | Conc. | μg · eqxhr/ | % |
| FORMULATIONS | [mg/kg] | [mg/ml] | ml | ABS* |
| TPGS + Pharmasolve (1.5:1) | 8.2 | 25 | 8.93 | 35.2 |
| TPGS + Pharmasolve (1:1) | 9.5 | 25 | 8.72 | 29.8 |
| TPGS + Pharmasolve (2:1) | 9.1 | 25 | 8.83 | 31.4 |
| TPGS + Propylene glycol | 8.5 | 20 | 9.65 | 36.9 |
| (1:1) | | | | |
| TPGS + Pharmasolve + PEG | 9.0 | 25 | 8.31 | 29.8 |
| 200 (2:1:1) | | | | |
| TPGS + Pharmasolve + PEG | 8.2 | 25 | 6.62 | 26.3 |
| 400 (2:1:1) | | | | |
| TPGS + Pharmasolve + PG | 8.9 | 25 | 8.07 | 29.3 |
| (2:1:1) | | | | |
| TPGS + Mygliol 810 (1:1) | 9.1 | 25 | 5.65 | 20.0 |
| TPGS + Softigen 767 (1:1) | 10.2 | 25 | 8.66 | 27.5 |
| TPGS + PEG 200 (1:1) | 8.3 | 25 | 7.75 | 30.4 |
| TPGS + PEG 400 (1:1) | 9.6 | 25 | 7.32 | 24.6 |

Softigen 767: PEG-6-Caprylic/Capric Glycerides
POE and Hydrogenated Castor Oil Derivatives as Carriers

Table 14 summarizes data for formulations containing POE and hydrogenated castor oil derivatives as carriers. The data set forth correspond to the data described in Example 7.

TABLE 14
Absorption Results of Polyoxyethylated Castor Oil
(Cremophor) Derivative Systems as Carriers

| | | | AUC | |
|---|---|---|---|---|
| | Dose | Conc. | μg · eqxhr/ | % |
| FORMULATIONS | [mg/kg] | [mg/ml] | ml | ABS |
| IV Paxene | 10.0 | 6 | 11.15 | 37.2 |
| Cremophor EL + Ethanol + | 9.2 | 1.3 | 6.07 | 21.5 |
| Water (1:1:8) | | | | |
| IV Paxene + Water (1:1) | 8.9 | 3 | 8.70 | 31.8 |
| IV Paxene + Water (1:5) | 9.1 | 1 | 10.76 | 38.5 |
| Cremophor EL + Pharmasolve | 8.6 | 20 | 6.74 | 25.3 |
| (1:1) | | | | |
| Cremophor EL + TBC (1:1) | 9.0 | 20 | 9.35 | 31.9 |
| Cremophor EL + Gelucire 44/14 | 9.8 | 25 | 11.5 | 38.1 |
| (1:6) | | | | |
| Cremophor EL + Gelucire 50/13 | 9.1 | 25 | 8.11 | 28.9 |
| (1:6) | | | | |
| Cremophor RH 40 + Ethanol + | 9.0 | 3 | 7.14 | 25.7 |
| Water (1:1:2) | | | | |
| Cremophor RH 40 + Gelucire | 9.5 | 25 | 10.7 | 36.6 |
| 44/14 (1:6) | | | | |
| Cremophor RH 40 + Gelucire | 9.1 | 20 | 7.73 | 27.5 |
| 44/14 + Olive Oil (1:2:1) | | | | |

Polysorbate 80 Carriers

Table 15 summarizes data for formulations containing polysorbate 80 as at least one of the carriers. The data set forth correspond to the data described in Example 7.

TABLE 15
Absorption Results of Polysorbate 80
(Tween 80) Systems as Carriers

| | Dose | Conc. | AUC μg · | % |
|---|---|---|---|---|
| FORMULATIONS | [mg/kg] | [mg/ml] | eqxhr/ml | ABS |
| Polysorbate 80 | 9.0 | 18 | 11.4 | 40.9 |
| Polysorbate 80 + Ethanol + | 8.0 | 1.2 | 7.92 | 31.2 |
| Water (1:1:8) | | | | |
| Polysorbate 80 + Ethanol (3:1) | 8.9 | 18 | 9.97 | 36.3 |
| Polysorbate 80 + Water (3:1) | 8.2 | 18 | 7.15 | 28.3 |
| Polysorbate 80 + TBC (1:1) | 9.5 | 20 | 9.12 | 31.2 |
| Polysorbate 80 + ATEC (1:1) | 9.1 | 20 | 8.50 | 30.3 |
| Polysorbate 80 + Olive oil (3:1) | 9.0 | 20 | 13.3 | 43.7 |
| Polysorbate 80 + PEG 400 (1:1) | 9.7 | 20 | 9.41 | 31.5 |
| Polysorbate 80 + Gelucire | 9.6 | 20 | 11.9 | 39.9 |
| 44/14 + Olive Oil (1:2:1) | | | | |
| Polysorbate 80 + Gelucire | 9.7 | 25 | 10.05 | 33.5 |
| 44/14 (1:6) | | | | |

TBC = Tributyl citrate (citrate ester)
ATEC = Acetyl triethyl citrate (citrate ester)

It has thus been shown that there are provided compositions and methods which achieve the various objects of the invention and which are well adapted to meet the conditions of practical use. As various possible embodiments might be made of the above invention, and as various changes might be made in the embodiments set forth above, it is to be understood that all matters herein described are to be interpreted as illustrative and not in a limiting sense.

As used herein, the term “about” is intended to convey that the numbers and ranges disclosed herein are flexible and that practice of the present invention using temperatures, concentrations, amounts, etc. outside of the range or different from a single value will achieve the desired result. The term typically includes a deviation of ±10% of any value it modifies.

The present invention is useful in clinical medicine, and particularly in the treatment of malignant and non-malignant diseases.

1. A composition comprising a taxane, a carrier comprising vitamin E TPGS, a co-solubilizer comprising ethanol in an amount of at least about 5% by weight of the composition, and propylene glycol, and a stabilizer, wherein said composition is in a form suitable for oral administration to a mammal.

2. The composition of claim 1, wherein said taxane is paclitaxel or docetaxel.

3. The composition of claim 1, wherein said taxane is present in a concentration of from about 2 to about 100 mg/ml.

4. The composition of claim 3, wherein the concentration of said taxane is from about 10 to about 50 mg/ml.

5. The composition of claim 1, wherein said carrier further comprises saturated polyglycolyzed glycerides, modified castor oils, polyoxyethylated stearate esters, polyoxyethylated sorbitan esters, polyoxyethylated fatty ethers, modified almond and corn oil glycerides, sorbitan diisostearate esters, polyoxyethylated hydroxystearates and cyclodextrin.

6. The composition of claim 1, wherein the ethanol is dehydrated.

7. The composition of claim 1, further comprising a surfactant.

8. The composition of claim 7, wherein said surfactant is dl-alpha-tocopherol or beta-carotene.

9. The composition of claim 8, comprising from about 2 mg/g (0.2%) to about 10 mg/g (1.0%) by weight of said dl-alpha-tocopherol.

10. The composition of claim 1, wherein said stabilizer is ascorbyl palmitate.

11. The composition of claim 1, wherein the stabilizer is dl-alpha-tocopherol.

12. The composition of claim 1, wherein the stabilizer is a radical inhibitor.

13. The composition of claim 1, further comprising a pharmaceutical excipient, diluent, sweetener, flavoring agent and/or coloring agent.

14. The composition of claim 1 further comprising a bioavailability-enhancing agent.

15. The composition of claim 14, wherein said bioavailability enhancing agent is a cyclosporin.

16. The composition of claim 1, wherein said ethanol is dehydrated.

17. The composition of claim 1, wherein said polypropylene glycol is present in an amount of from about 35 to about 40% by weight.

18. The composition of claim 1, wherein said taxane comprises paclitaxel present in an amount of about 1.2% by weight, wherein said Vitamin E TPGS is present in an amount of about 40% by weight, wherein said ethanol is present in an amount of about 18% by weight of said composition wherein said propylene glycol is present in an amount of about 40% by weight, said stabilizer comprises ascorbyl palmitate present in an amount of about 0.5% by weight, and wherein said composition further comprises dl-alpha-tocopherol in an amount of about 0.5% by weight.

19. The composition of claim 1, wherein said taxane comprises paclitaxel present in an amount of about 1.2% by weight, wherein said Vitamin E TPGS is present in an amount of about 40% by weight, wherein said ethanol is present in an amount of about 22% by weight of said composition wherein said propylene glycol is present in an amount of about 35% by weight, said stabilizer comprises ascorbyl palmitate present in an amount of about 0.5% by weight, and wherein said composition further comprises dl-alpha-tocopherol in an amount of about 0.5% by weight.

20. A method to achieve target blood levels of a taxane in a mammal comprising orally administering to said mammal a pharmaceutical composition comprising a taxane, a carrier comprising vitamin E TPGS, a co-solubilizer comprising ethanol in an amount of at least about 5% by weight of the composition, and propylene glycol, and a stabilizer.

21. A method treating a mammalian subject suffering from a taxane-responsive disease comprising the step of orally administering to said mammal a pharmaceutical composition comprising a taxane, a carrier comprising vitamin E TPGS, a co-solubilizer comprising ethanol in an amount of at least about 5% by weight of the composition, and propylene glycol, and a stabilizer.
